(19) United States
(12) Patent Application Publication
(10) Pub. No.: US 2013/0025197 A1
(43) Pub. Date: Jan. 31, 2013

(54) Reduction and induction of protein folding

(71) Applicant: Gabriel James Tambunga, Philadelphia, PA (US)

(72) Inventor: Gabriel James Tambunga, Philadelphia, PA (US)

(21) Appl. No.: 13/136,285

(22) Filed: Jul. 29, 2011

(51) Int. Cl. A01H 3/00; A01K 29/00

(52) U.S. Cl. 47/58.1 R; 119/712

(57) ABSTRACT

The Reduction and Induction of Protein Folding is a process where isotopes of atoms that compose a protein are replaced by other isotopes that can either enhance proper or improper protein folding in a living organism. To enhance proper protein folding, atoms or molecules with isotopes with typically the same number of protons and neutrons can be applied to a living organism through a created environment. To enhance improper protein folding, atoms or molecules with isotopes with typically different number of protons and neutrons can be applied to a living organism through a created environment. The isotopes in an atom or molecule can be accumulated and created artificially, respectively, where the living organism consumes the accumulated atoms or created molecules where eventually the living organism will only contain the isotopes or molecules that contain the desired isotopes.

### BACKGROUND OF THE INVENTION

1. Field of Invention

The present invention relates generally to a process of reducing or inducing improper protein folding by eliminating or increasing undesired and desired atomic isotopes, respectively.

2. Discussion of Prior Art

Improper protein folding can lead to protein folding diseases. These diseases include Alzheimer's disease, Prion diseases, and types of cancer. There is no prior art that relates heavy isotopes to improper folding. However, it is well known that the most typical and stable atoms, which compose all known mass, are those that contain nuclei that is composed of neutrons and protons that are equal in number. For example, the most common and most stable carbon dioxide molecule are carbon dioxide molecules that contain carbon with 6 neutrons and 6 protons. A change in an isotope in a molecule can produce a small change in its chemical behavior. According to Jochen Hoefs, Stable Isotope Geochemistry, Springer 2009, Page 4, “The replacement of any atom in a molecule by one of its isotopes produces a very small change in chemical behavior. The addition of one neutron can, for instance, considerably depress the rate of chemical reaction.” This would include proteins and its effect on protein folding. Proteins fold and connect at specific points on the protein chain. If the isotopes of the atoms change at about the locations where the connections occur, for example, the folding of the protein can be affected. There has been some interest in reducing improper protein folding.

U.S. Patent No. 20070037258 discusses methods and compositions for enhancing protein folding. U.S. Pat. No. 6,060,293 discusses the use of electromagnetic radiation to enhance the proper folding and improper folding of proteins. U.S. Patent No. 20080014191A1 discusses the use of agents to reduce protein mis-folding.

The reduction of undesired isotopes can enhance proper protein folding, which can result in the reduction of protein folding disease. Furthermore, an increase of desired isotopes, such as heavier isotopes for example, can result in an increase in improper folding that can lead to protein folding diseases.

### REFERENCES CITED

### U.S. Patent Documents

| | | |
|---|---|---|
| 20070037258 | February 2007 | Snyder |
| 6,060,293 | May 2000 | Bohr |
| 20080014191A1 | January 2008 | Balch |

### Other Publications

Hoefs, Jochen: Stable Isotope Geochemistry; Springer 2009, Page 4.

### SUMMARY OF THE INVENTION

The purpose of this process is to reduce the number of incorrect protein folding in a living organism or to increase the number of incorrect protein folding. To reduce the amount of incorrect protein folding includes reducing the number of unwanted isotopes that constitute the protein. To increase the amount of incorrect protein folding includes reducing the number of isotopes that allow proper protein folding.

The process of reducing the amount of incorrect protein folding includes: (1) determining that proper protein folding is desired; (2) determining what is consumed by the living organism for growth or survival, such as a gas, liquid, and/or waves (i.e. electromagnetic radiation); (3) determining all atoms, molecules, and/or waves involved in the consumption; (4) determining the isotopes that will enhance proper protein folding; (5) creating and/or acquiring the isotopes that will enhance proper protein folding; (6) acquiring or, with isotopes in (5), creating the molecules used in consumption containing isotopes that will enhance proper protein folding; (7) determining a way to artificially generate the waves in (3); (8) creating an environment where consumption of the living organism is controlled, and an optimal environment for growing or sustaining the living organism is maintained; (9) applying all created and/or acquired atoms and/or molecules and/or waves to the controlled environment; (10) placing the living organism in the controlled environment so proper protein folding can occur.

The process of reducing the amount of correct protein folding includes: (1) determining that improper protein folding is desired; (2) determining what is consumed by the living organism for growth or survival, such as a gas, liquid, and/or waves (i.e. electromagnetic radiation); (3) determining all atoms, molecules, and/or waves involved in the consumption; (4) determining the isotopes that will enhance improper protein folding; (5) creating and/or acquiring the isotopes that will enhance improper protein folding; (6) acquiring or, with isotopes in (5), creating the molecules used in consumption containing isotopes that will enhance improper protein folding; (7) determining a way to artificially generate the waves in (3); (8) creating an environment where consumption of the living organism is controlled, and an optimal environment for growing or sustaining the living organism is maintained; (9) applying all created and/or acquired atoms and/or molecules and/or waves to the controlled environment; (10) placing the living organism in the controlled environment so improper protein folding can occur.

### DESCRIPTION OF PROCESS

The following is the description of the process of increasing or decreasing the isotopes that will enhance proper or improper protein folding. The process is broken down into steps where the steps apply to a desire to increase proper protein folding or decrease proper protein folding.
(a) Determine if proper or improper protein folding is desired for a specific protein or proteins within a living organism.
(b) Determine what is consumed to grow and/or sustain the life of the living organism.
(c) Determine all atoms, molecules and/or waves used in consumption in (b).
(d) Determine the isotopes for the atoms, molecules and/or waves indicated in (c) that will enhance proper protein folding if proper protein folding is desired. Determine the isotopes for the atoms, molecules and/or waves indicated in (c) that will enhance improper protein folding if improper protein folding is desired.
(e) Create and/or acquire the atoms in (d).
(f) Acquire or, with atoms in (e), create all molecules indicated in (d).
(g) Determine a way to artificially generate the waves that are consumed.
(h) Create an environment where the living organism can maintain existence or grow, and can only consume created atoms and molecules, (e) and (f), respectively.
(i) Apply atoms in (e) and molecules in (f), and waves in (g) to the created environment (h).
(j) Place living organism in the created environment, and increase or decrease concentrations of created atoms and molecules for optimal growth or survival with desired proteins.

Due to different types of living organisms: not all steps will be necessary; not all steps will be needed in the specific order; some steps may be repeated.

### BRIEF DESCRIPTION OF THE DRAWINGS

FIG. 1 is a side cross sectional view of a created environment that will allow proper protein folding for an immature plant (i.e. a seed).

FIG. 2 is a side cross sectional view of a created environment that will allow proper protein folding for an herbivore.

FIG. 3 is a side cross sectional view of a created environment that will allow proper protein folding for an omnivore.

### DETAILED DESCRIPTION OF PREFERRED EVENTS

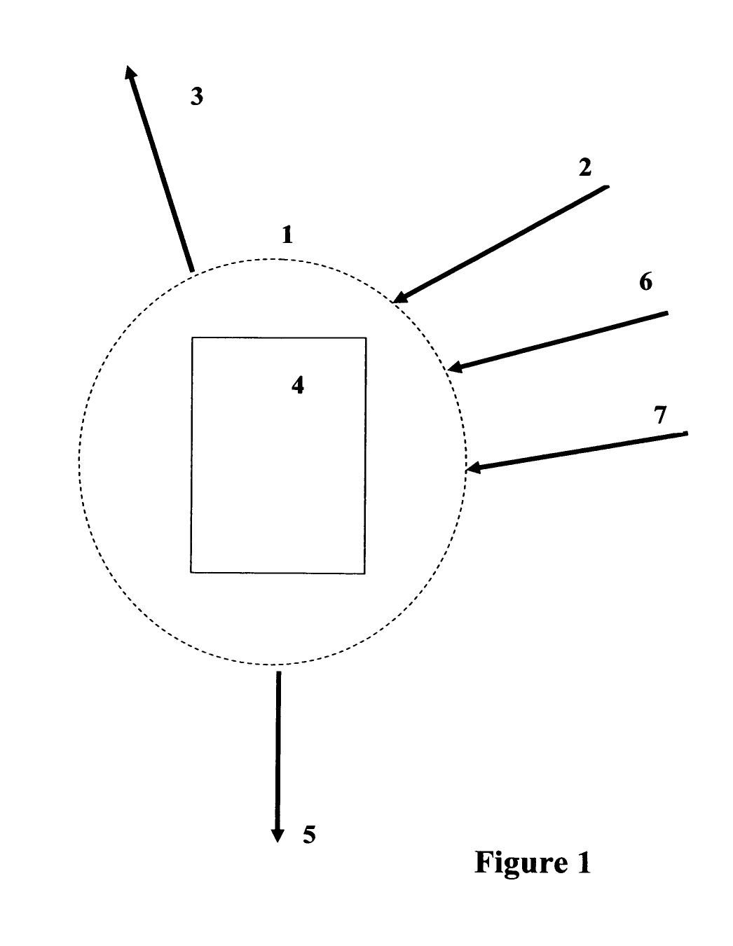

With reference now to the drawings, and particularly to FIG. 1, there is shown a cross sectional view of a created environment 1 for a plant 4. The process of causing plant 4 to grow with isotope controlled soil nutrients 6, absorbed gas 2, and electromagnetic radiation 7 includes the following steps.
(a) Proper protein folding is desired for a specific protein.
(b) It is determined that soil nutrients 6, absorbed gas 2 and electromagnetic radiation, 7, is used for plant growth, 4.
(c) The following atoms (with atomic symbols) are needed for soil nutrients 6 and absorbed gas 2: Nitrogen (N), Phosphorous (P), Potassium (K), Calcium (Ca), Magnesium (Mg), Sulphur (S), Iron (Fe), Manganese (Mn), Zinc (Zn), Copper (Cu), Boron (B), Chlorine (Cl) and Molybdenum (Mo), Carbon (C), Oxygen (O), Hydrogen (H). The soil nutrients 6 and absorbed gas 2 information was acquired from: Guide to Symptoms of Plant Nutrient Deficiencies by the University of Arizona Cooperative Education 1999; and Nutrient Deficiencies & Toxicities in Crop Plants, W. F. Bennett, APS Press, 1993. The molecules that will introduce the atoms to the plants are: for N create nitrate (NO₃), for P create Phosphate (PO₄), for K create Sulphate (K₂SO₄), for Ca create Calcium Carbonate (CaCO₃), for Mg create magnesium Sulfate (MgSO₄), for Fe create Iron Sulfate (FeSO₄), for B create Borate (BO3), for Mo create Molybdate (MoO₄). An additional soil nutrient 6 would be H₂O. The gas 2 to be introduced is CO₂. The remaining atoms can be applied directly into plants. It was determined from K. Okamoto et al, Development of Plant Growth Apparatus Using Blue and Red LED As Artificial Light Source, ISHS Acta Horticulturae 440: International Symposium on Plant Production in Closed Ecosystems that an artificial light source can be used, where the artificial light source 7 will contain LED's with emissions at wavelengths 450 nm and 660 nm.
(d) As the most common isotopes are atoms with the same number neutrons as protons the following isotopes will be used for plant growth: ¹⁴N, ³⁰P, ³⁸K, ⁴⁰Ca, ²⁴Mg, ³²S, ⁵²Fe, ⁵⁰Mn, ⁶⁰Zn, ⁵⁸Cu, ¹⁰B, ³⁴Cl, ⁸²Mo, ¹²C, ¹⁶O and ¹H.
(e) Acquire the isotopes indicated in (d). The isotopes in (d) can be separated from other isotopes by the use of lasers as indicated by: P. A. Bokan et al, Laser Isotope Separation in Atomic Vapor, Wiley-VCH Verlag GmbH & Co., 2006; and C. B. Moore, The Application of Lasers to Isotope Separation, Accounts of Chemical Research, Vol. 6, pages 323-328, 1973.
(f) Using chemistry create the molecules 6 in (c) that will introduce the atoms in (d) to plants.
(g) Acquire LED sources 7 with emissions at wavelengths 450 nm and 660 nm.
(h) An isolated chamber 1 will be created to introduce the created CO₂ gas 2, created nutrients 6, LED sources 7, to expel gas 3 and any excretions from the plant and/or unwanted excesses can exit through 5, and maintain temperature that is optimal for growth. The concentration of atoms and compounds may vary per plant type. Create station in chamber to hold seed of plant, to be sprayed by nutrients 6, and to be adequately illuminated by LED light source 7 for optimal plant growth. The soil nutrients can be placed in water and introduced to the plant via a spray head, tubing and a pump (i.e. an aeroponics system).
(i) Activate power for LED light source 7, and aeroponics system in chamber 1.
(j) Place seed of plant, 4, in chamber 1. Increase or decrease concentrations of liquid nutrients, gas or light source as needed for optimal plant growth.

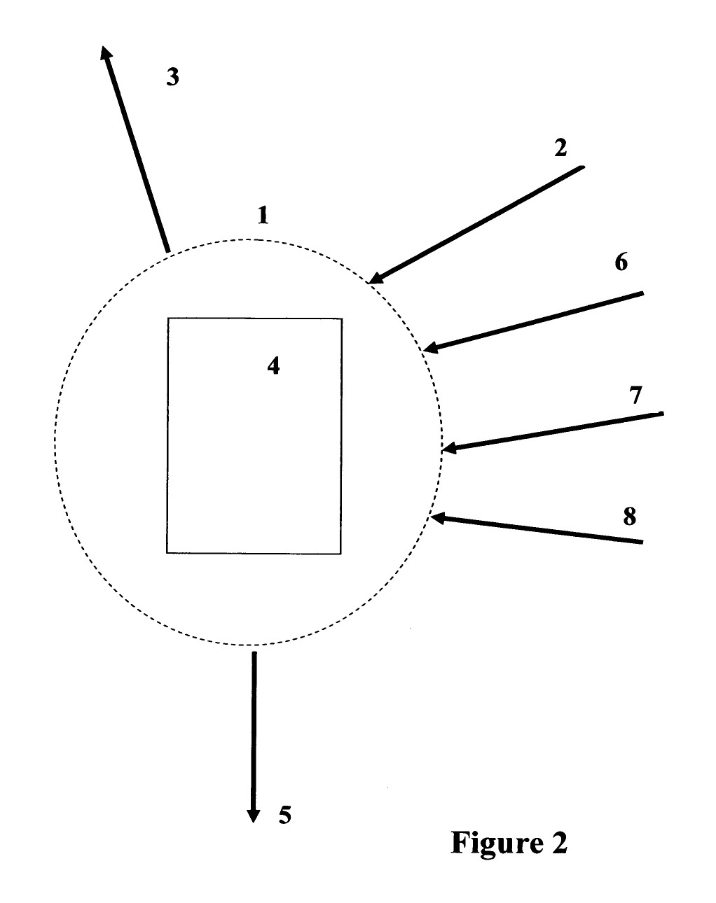

With reference now to the drawings, and particularly to FIG. 2, there is shown a cross sectional view of a created environment 1 for an herbivore 4. The process of causing an herbivore 4 to grow with isotope controlled created plants 6, absorbed gas (air) 2, water 7 and a light source 8 includes the following steps.
(a) Proper protein folding is desired for specific proteins.
(b) It is determined that plants 6, air 2, water 7, and a light source 8 are used for herbivore growth 4.
(c) Water 7 is created from H₂0. Air 2 is composed of Nitrogen (N), Oxygen (O), Argon (Ar), Carbon Dioxide (CO₂), Neon (Ne), Helium (He), Methane (CH₄), Krypton (Kr), Hydrogen (H) and Xenon (Xe). In air Nitrogen is introduced as N₂, Oxygen as O₂, and Hydrogen as H₂. The following atoms (with atomic symbols) are needed for water and air: Nitrogen (N) Oxygen (O), Argon (Ar), Carbon (C), Neon (Ne), Helium (He), Krypton (Kr), Hydrogen (H), and Xenon (Xe). He, Ar, Ne, Kr, and Xe can be introduced to the herbivore as individual atoms. Herbivores acquire its nutrients from plants 6, where the plants 6 vary due to herbivore diet and its atomic and molecular composition. The plants 6 specific to the herbivores diet will be grown to have specific isotopes as the previous detailed description associated with FIG. 1.
(d) As the most common isotopes are atoms with the same number neutrons as protons the following isotopes will be used for water 7 and air 2: ¹⁴N, ¹⁶O, ³⁶Ar, ¹²C, ²⁰Ne, ⁴He, ⁷²Kr, ¹H, and ¹⁰⁸Xe.
(e) Acquire the isotopes indicated in (d). The isotopes in (d) can be separated from other isotopes by the use of lasers as indicated by: P. A. Bokan et al, Laser Isotope Separation in Atomic Vapor, Wiley-VCH Verlag GmbH & Co., 2006; and C. B. Moore, The Application of Lasers to Isotope Separation, Accounts of Chemical Research, Vol. 6, pages 323-328, 1973.
(f) Using chemistry, create water (H₂O) 7 with atoms in (e). Using chemistry create the molecules in (c) that will be used as air 2 with atoms in (e). The plants 6 specific to the herbivores diet will be grown to have specific isotopes as the previous detailed description associated with FIG. 1.
(g) Acquire an incandescent light source 8, where the light source 8 is for comfort of the living organism.
(h) An isolated chamber 1 with an artificial light source 8 will be created. Air will be introduced to the chamber 2 and allow excess gas excretions to exit the chamber through 3. The chamber 1 will allow other excretions to exit through 5 to not allow the reintroduction of the excretions to the animal diet. The created water will be introduced through 7 and created plants 6 will be accessible to the herbivore 4 in the chamber 1. The chamber 1 will contain an electric heat source that is optimal for the survival of the living organism.
(i) Activate light source 8, air source 2, water source 7 and grown plants 6 in chamber 1.
(j) Place young herbivore 4 in chamber 1. Increase or decrease concentrations of air 2, water 7, light 8 and created plants 6 for optimal herbivore growth.

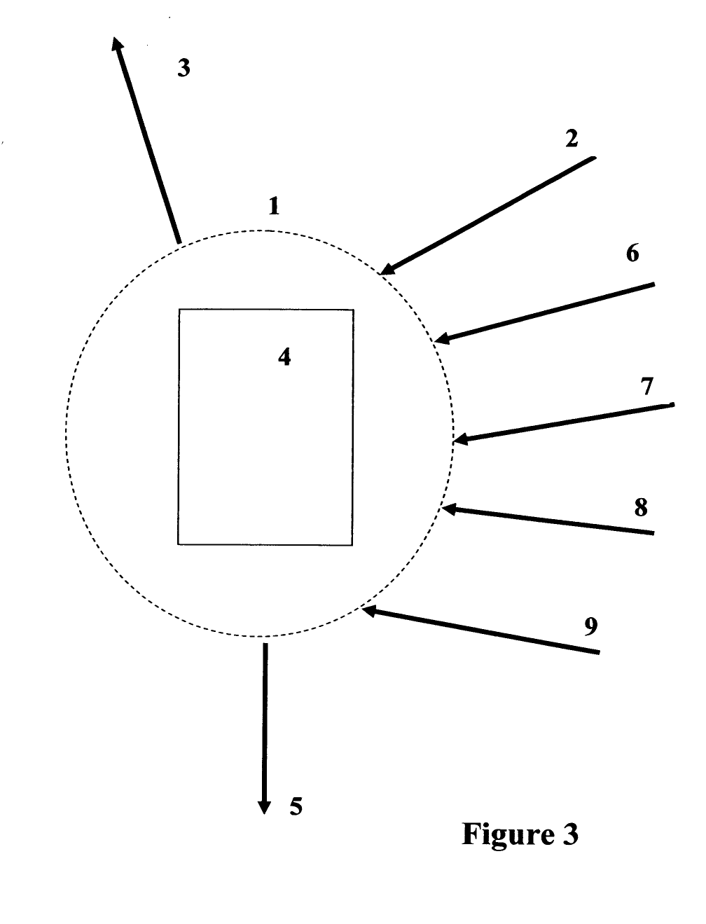

With reference now to the drawings, and particularly to FIG. 3, there is shown a cross sectional view of a created environment 1 for an omnivore 4. The process of causing an omnivore 4 to grow with isotope controlled plants 6, herbivore meat 9, absorbed gas (air) 2, water 7 and a light source 8 includes the following steps
(a) Proper protein folding is desired.
(b) It is determined that plants 6, herbivore meat 9, air 2, and water 7, are used for omnivore growth 4.
(c) Water 7 is created from H₂0. Air 2 is composed of Nitrogen (N), Oxygen (O), Argon (Ar), Carbon Dioxide (CO₂), Neon (Ne), Helium (He), Methane (CH₄), Krypton (Kr), Hydrogen (H) and Xenon (Xe). In air Nitrogen is introduced as N₂, Oxygen as O₂, and Hydrogen as H₂. The following atoms (with atomic symbols) are needed for water and air: Nitrogen (N) Oxygen (O), Argon (Ar), Carbon (C), Neon (Ne), Helium (He), Krypton (Kr), Hydrogen (H), and Xenon (Xe). He, Ar, Ne, Kr, and Xe can be introduced to the herbivore as individual atoms. Omnivores acquire its nutrients from herbivore meat 9 and plants 6, where the herbivore meat 9 and plants 6 vary due to omnivore diet and their atomic and molecular composition. The plants 6 specific to the omnivore's diet will be grown to have specific isotopes as the previous detailed description associated with FIG. 1. The herbivore meat 9 specific to the omnivores diet will be grown to consume specific plants that have specific isotopes as the previous detailed description associated with FIG. 2.
(d) As the most common isotopes are atoms with the same number neutrons as protons the following isotopes will be used for water 7 and air 2: ¹⁴N, ¹⁶O, ³⁶Ar, ¹²C, ²⁰Ne, ⁴He, ⁷²Kr, ¹H, and ¹⁰⁸Xe.
(e) Acquire the isotopes indicated in (c). The isotopes in (c) can be separated from other isotopes by the use of lasers as indicated by: P. A. Bokan et al, Laser Isotope Separation in Atomic Vapor, Wiley-VCH Verlag GmbH & Co., 2006; and C. B. Moore, The Application of Lasers to Isotope Separation, Accounts of Chemical Research, Vol. 6, pages 323-328, 1973.
(f) Using chemistry, create water (H₂O) 7 with atoms in (e). Using chemistry create the molecules in (c) that will be used as air 2 with atoms in (e). The plants 6 specific to the omnivore's diet will be grown to have specific isotopes as the previous detailed description associated with FIG. 1. The herbivore meat 9 specific to the omnivores diet will be grown to consume specific plants that have specific isotopes as the previous detailed description associated with FIG. 2.
(g) Acquire an incandescent light source 8, where the light source 8 is for comfort of the living organism.
(h) An isolated chamber 1 with an artificial light source 8 will be created. Air will be introduced to the chamber 2 and allow excess gas excretions to exit the chamber through 3. The chamber 1 will allow other excretions to exit through 5 to not allow the reintroduction of the excretions to the animal diet. The created water will be introduced through 7, herbivore meat 9 and created plants 6 will be accessible to the omnivore 4 in the chamber 1. The chamber 1 will contain an electric heat source that is optimal for the survival of the living organism.
(i) Activate light source 8, air source 2, water source 7, omnivore meat 9 and grown plants 6 in chamber 1.
(j) Place young omnivore 4 in chamber 1. Increase or decrease concentrations of air 2, water 7, light 8, omnivore meat 9 and created plants 6 for optimal omnivore growth.

While particular embodiments of the process have been shown and described, it will be obvious to those skilled in the art that changes and modifications may be made without departing from the process in its broader aspects that fall within the true spirit and scope of the process. All detailed descriptions and drawings discussed can apply to the desire to increase improper folding by substituting in (a) Proper protein folding is desired to Improper protein folding is desired, and changing the isotopes in (d) to isotopes that can increase improper folding such as heavier isotopes.

I claim:

1. A method of increasing proper protein folding, comprising the steps of:
determining if said proper protein folding is desired for a specific protein(s) within a living organism;
determining what is consumed to grow or sustain the said living organism;
determining all atoms, molecules and waves used in what is said consumed;
determining the isotopes of the said atoms, said molecules and said waves in what is said consumed that will said increase proper protein folding;
acquiring or creating the said isotopes of the said atoms in what is said consumed that will said increase proper protein folding;
acquiring or creating all said molecules in what is said consumed that will said increase proper protein folding with said isotopes of the said atoms in what is said consumed that will said increase proper protein folding;
generating artificially the said waves used in what is said consumed;
creating an environment where the said living organism can said grow or sustain optimally, and where the said living organism can only consume acquired or created said isotopes of the said atoms in what is said consumed that will said increase proper protein folding, and acquired or created said molecules in what is said consumed that will said increase proper protein folding;
applying said acquired or created isotopes and said created molecules and said waves to the created said environment;
placing said living organism in said created environment.

2. A method of increasing improper protein folding, comprising the steps of:
determining if said improper protein folding is desired for a specific protein(s) within a living organism;
determining what is consumed to grow or sustain the said living organism;
determining all atoms, molecules and waves used in what is said consumed;
determining the isotopes of the said atoms, said molecules and said waves in what is said consumed that will said increase improper protein folding;
acquiring or creating the said isotopes of the said atoms in what is said consumed that will said increase improper protein folding;
acquiring or creating all said molecules in what is said consumed that will said increase improper protein folding with said isotopes of the said atoms in what is said consumed that will said increase improper protein folding;
generating artificially the said waves used in what is said consumed;
creating an environment where the said living organism can said grow or sustain optimally, and where the said living organism can only consume acquired or created said isotopes of the said atoms in what is said consumed that will said increase improper protein folding, and acquired or created said molecules in what is said consumed that will said increase improper protein folding;
applying said acquired or created isotopes and said created molecules and said waves to the created said environment;
placing the said living organism in said created environment.
